(19) United States
(12) Patent Application Publication
(10) Pub. No.: US 2005/0121346 A1
(43) Pub. Date: Jun. 9, 2005

(54) Mountable container apparatus for patient transport device and methods regarding same

(75) Inventors: Daniel L. Cummings, Wayzata, MN (US); Jacob D. Cummings, St. Michael, MN (US); Beverly J. Ott, Rochester, MN (US)

Correspondence Address: MUETING, RAASCH & GEBHARDT, P.A., P.O. BOX 581415, MINNEAPOLIS, MN, 55458, US

(73) Assignee: PHS West Incorporated, Hanover, MN (US)

(21) Appl. No.: 11/002,941

(22) Filed: Dec. 2, 2004

Related U.S. Application Data
(60) Provisional application No. 60/616,996, filed on Oct. 8, 2004.
(60) Provisional application No. 60/527,375, filed on Dec. 5, 2003.

(51) Int. Cl. A61L 15/00

(52) U.S. Cl. 206/438

(57) ABSTRACT

Apparatus and methods for use with a transport device (e.g., a stretcher) include a closable outer container mountable to the transport device and a single use inner container configured to be retained within the outer container. The inner container defines an opening through which objects associated with a person may be inserted into the single use inner container. The single use inner container further includes a tamper evident closure for use in sealing the opening thereof.

### CROSS-REFERENCE TO RELATED APPLICATIONS

This application claims the benefit of U.S. Provisional Application Ser. No. 60/527,375 filed 5 Dec. 2003 and U.S. Provisional Application Ser. No. 60/616,996 filed 8 Oct. 2004, which are both incorporated herein by reference in their entirety.

### BACKGROUND OF THE INVENTION

The present invention relates to transport devices (e.g., patient transport devices such as gurney's, stretchers, beds, etc.). More particularly, the present invention pertains to the movement of a person's belongings along with such transport devices.

Various methods are available for movement of a person (e.g., a patient) and/or secure movement of a person's personal affects in one or more settings (e.g., outpatient surgery, emergency room environments, long term care facilities, movement of a patient from floor to floor in a hospital, etc.). For example, U.S. Pat. No. 5,005,230 to Congdon, entitled “Patient Transporter,” issued 9 Apr. 1991, describes the transport of a patient's belongings along with the patient using a patient transport system. The provision of locked containers and the use of disposable plastic bags are described therein.

Further, for example, other ways of securing the personal belongings of a person (e.g., a patient) are also used in one or more situations such that the person's personal property is not lost, misplaced, soiled, or stolen while the person is away from such belongings. For example, lockers or other check-in systems have been used for securing a person's personal property.

### SUMMARY OF THE INVENTION

The present invention, at least in one embodiment, reduces the risk of a person's personal property, when disassociated therefrom, from being lost, misplaced, soiled, or stolen. Further, the present invention, at least in one embodiment, is designed to provide for security of such a person's belongings and information, thereby minimizing potential institutional liability for the loss of, for example, a patient's personal items or patient information. In other words, the present invention, at least in one embodiment, is provided to assist in protecting a staff, institution, and a patient from loss of personal belongings and patient information (e.g., protect a person's privacy). One or more of the embodiments of the present invention may be employed to achieve one or more of the benefits or advantages described herein.

A container apparatus for use with a transport device (e.g., a stretcher, a gurney, an examination cart, a wheelchair), according to one embodiment of the present invention, includes a closeable outer container mountable to the transport device. Further, the container apparatus includes at least one single use inner container configured to be retained within the outer container. The at least one single use inner container defines an opening through which objects associated with a person may be inserted into the single use inner container. The at least one single use inner container includes a tamper evident closure for use in sealing the opening.

In one embodiment of the container apparatus, the closeable outer container may be lockable to secure the at least one single use inner container within the closeable outer container. Further, for example, the container apparatus may include a keylock for locking the closeable outer container. The keylock being locked only when a key thereof is removed. Such a key may be secured to the person who owns the belongings.

In another embodiment of the container apparatus, the closeable outer container includes a first body of material defining a cavity configured to receive the at least one single use inner container. Further, the closeable outer container may include a second body of material extending from the first body of material to a distal end thereof with a mounting device associated with the distal end of the second body of the material for use in mounting the closeable outer container to the transport device (e.g., using a hook and loop fastener mounting device).

Yet further, in another embodiment of the container apparatus, the mounting device associated with the distal end of the second body of material may be configured to position the closeable outer container in a first state at least partially under a surface of the transport device when mounted thereto and when the outer container is closed. Further, the mounting device is configured to allow the closeable outer container to be repositioned to a second state at least partially above the surface of the transport device such that the cavity defined by the first body of material is accessible.

Still further, the container apparatus may further include an attachment mechanism adapted to secure the closeable outer container to the transport device after the closeable outer container is mounted thereto, and/or the single use inner container may include a handle (e.g., integrally formed with the container).

A method for securing objects belonging to a person when the person is transported using a transport device is also described. The method includes providing a closeable outer container mountable to the transport device. The method further includes inserting the objects of the person into at least one single use inner container configured to be retained within the closeable outer container. The at least one single use inner container includes a tamper evident closure for use in sealing the single use inner container. The at least one single use inner container is inserted within the closeable outer container (e.g., before or after the objects are inserted therein).

One or more embodiments of the method for securing such objects may further include one or more of the following features or steps: locking the closeable outer container (e.g., using a keylock); mounting the closeable outer container onto the transport device; providing a closeable outer container that includes a first body of material defining a cavity configured to receive at least one single use inner container, and a second body of material extending from the first body of material to a distal end thereof; positioning a first portion of a hook and loop fastener on a surface of a transport device, and thereafter attaching a second portion of a hook and loop fastener associated with the distal end of the second body of material to the first portion of the hook and loop fastener; mounting the closeable outer container onto the transport device such that the closeable outer container is in a first state at least partially under a surface of the transport device when mounted thereto; mounting the closeable outer container onto the transport device such that the closeable outer container is repositionable to a second state at least partially above the surface of the transport device such that a cavity defined within the closeable outer container is accessible (e.g., the closeable outer container rests on the surface of the transport device and the cavity is accessible); and securing the closeable outer container to the transport device after the closeable outer container is mounted thereto (e.g., using a lanyard).

A liner apparatus for use with a container apparatus mountable onto a transport device is also described. For example, the liner apparatus may include a body of liner material defining a cavity and an opening through which objects associated with a person may be inserted into the cavity. The body of liner material is configured to be retained within the mountable container apparatus when objects associated with a person are inserted into the cavity. The liner apparatus may further include a tamper evident closure for use in sealing the opening. In one or more embodiments, the liner apparatus may further include a handle, and the tamper evident closure may include a perforated tear strip.

The above summary of the present invention is not intended to describe each embodiment or every implementation of the present invention. Advantages, together with a more complete understanding of the invention, will become apparent and appreciated by referring to the following detailed description and claims taken in conjunction with the accompanying drawings.

### BRIEF DESCRIPTION OF THE DRAWINGS

FIG. 1 is a generalized block diagram of a personal object security system including a mountable container apparatus according to the present invention.

FIG. 2 is a block diagram showing one embodiment of a personal object security method according to the present invention that may be implemented using a personal object security system such as that shown in FIG. 1.

FIG. 3 is a block diagram of one exemplary security access technique as generally shown in the method of FIG. 2.

FIGS. 4A and 4B show perspective views of one exemplary embodiment of a personal object security system such as generalized in FIG. 1; wherein FIG. 4A shows a mountable container apparatus in a first state under a surface of the transport apparatus, and FIG. 4B shows the mountable container apparatus in a second state above the surface of the transport device ready for access by a person.

FIGS. 5A-5D show a first side view, a top plan view (with a portion thereof removed), a second side view, and a third side view (with a portion thereof removed) of one embodiment of the mountable container apparatus shown generally in FIGS. 4A and 4B.

FIG. 6 shows a perspective view of one exemplary embodiment of a single use inner container that may be used in a personal object security system such as that shown generally in FIG. 1.

FIG. 7 shows a top plan view of another exemplary embodiment of a single use inner container such that may be used in the personal object security system shown generally in FIG. 1.

FIGS. 8A and 8B show a perspective view of another alternate embodiment of a personal object security system according to the present invention, including a mountable container apparatus as shown in FIG. 8A along with a single use inner container as shown in FIG. 8B.

FIGS. 9A and 9B show a perspective view of another alternate embodiment of a personal object security system according to the present invention, including a mountable container apparatus as shown in FIG. 9A along with a single use inner container as shown in FIG. 9B.

FIGS. 10A and 10B show a perspective view of another alternate embodiment of a personal object security system according to the present invention mountable on a wheelchair, including a mountable container apparatus as shown in FIG. 10A along with a single use inner container as shown in FIG. 10B.

### DETAILED DESCRIPTION OF THE EMBODIMENTS

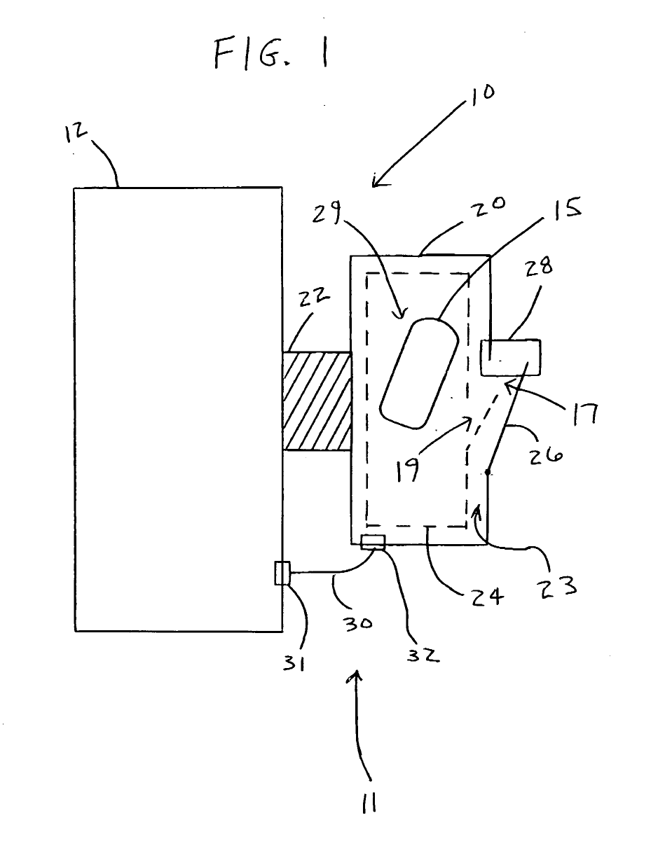

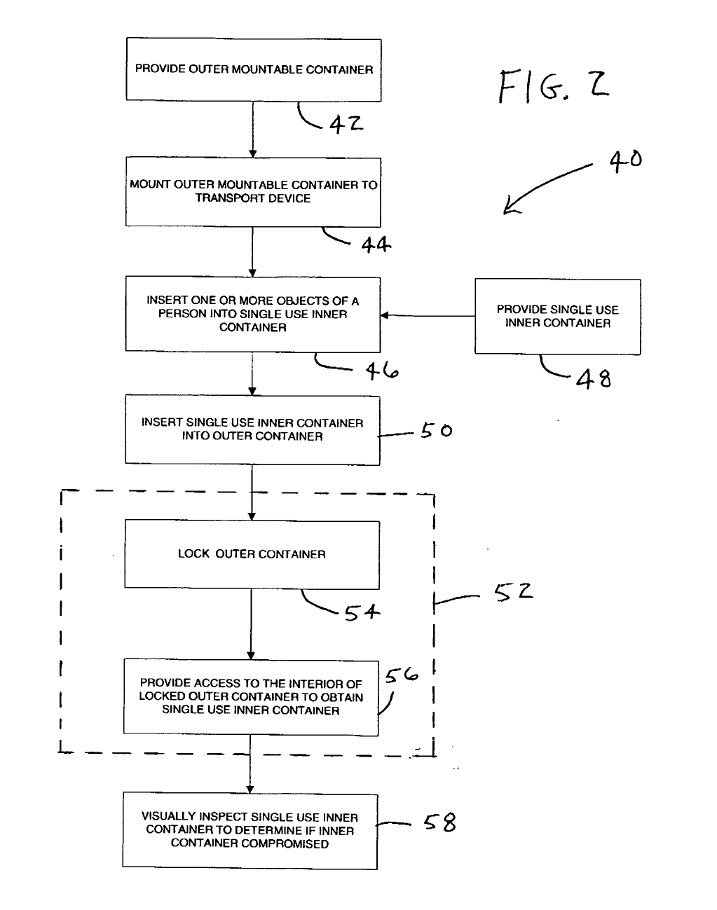

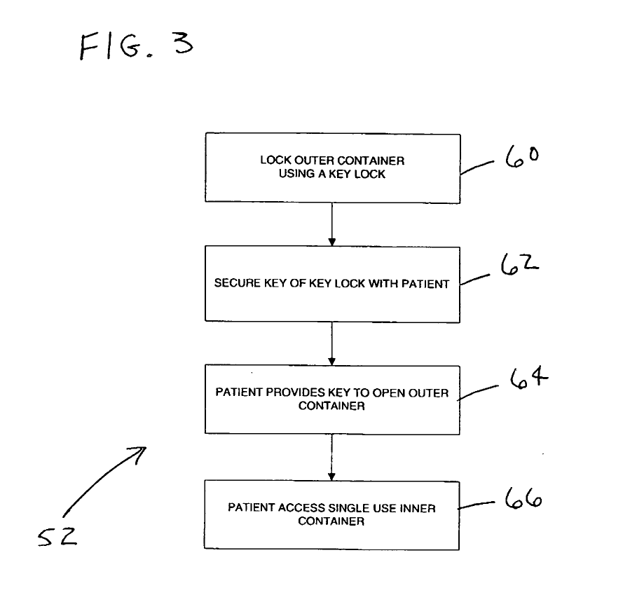

The present invention shall generally be described with reference to FIGS. 1-2. A more detailed description of one or more various embodiments of the present invention shall then be described with reference to FIGS. 3-10. It will be apparent to one skilled in the art that elements from one embodiment may be used in combination with elements of the other embodiments, and that the present invention is not limited to the specific embodiments described. Further, it will be recognized that the embodiments of the present invention described herein will include many elements that are not necessarily shown to scale. Further, it will be recognized that the size and shape of various elements herein may be modified without departing from the scope of the present invention, although one or more shapes and sizes may be advantageous over others.

FIG. 1 shows a generalized block diagram of a personal object security system 10 according to the present invention. The personal object security system 10, shown in the exemplary embodiment of FIG. 1, includes a mountable container apparatus 11. The mountable container apparatus 11 includes a mountable outer container apparatus 20 mounted to a transport device 12 by a mounting structure 22. The mountable outer container apparatus 20 defines a cavity 23 (e.g., a defined volume). Further, the mountable outer container 20 defines an opening 17 for access to the cavity 23. The mountable outer container 20 further includes a closure structure 26 for use in closing the opening 17. Further, a locking structure 28 is provided to secure the contents within cavity 23 (e.g., to lock the closure structure 26).

As further shown in FIG. 1, the mountable outer container 20 is configured to retain a single use inner container 24 (e.g., an inner container defining an inner volume 29 and an opening 19 such that objects 15 may be inserted therein) within cavity 23. Preferably, according to one embodiment of the present invention, the single use inner container includes a tamper evident closure for use in sealing the opening 19.

The transport device 12 may include any apparatus upon which the outer container 20 can be mounted using mounting structure 22. At least in one embodiment, the transport device 12 may include a device usable to transport a person (e.g., a patient or client). For example, the transport device 12 may include a gurney, a stretcher, a wheelchair, a cart, or any other apparatus used to move a person from one position to another. For example, such transport devices are used in hospitals, clinics, health care institutions, etc.

The mountable outer container 20 may be constructed of any material suitable for preventing access to objects (e.g., including access to the single use inner container 24) positioned within the cavity 23 thereof. For example, the outer container 20 may be formed of one or more materials such as vinyl, plastic, metal, wood, fiberglass, etc. Further, the mountable outer container 20 may be configured in one or more various shapes and sizes. Preferably, the mountable container is of a size adequate to retain the belongings of a patient in one or more settings (e.g., clothes, jewelry, private papers, wallets, shoes, and/or purses, belong to a patient at a hospital, clinic, health care institution, etc.).

The outer container 20 may be mounted to the transport device 12 by one or more various types of mounting structure 22. For example, the mounting structure 22 may include plastic or metal brackets, hook and loop fasteners (e.g., Velcro), bolts for attaching the outer container 20 onto a frame of a transport device, slide tracks for receiving the outer container on a transport device, etc.

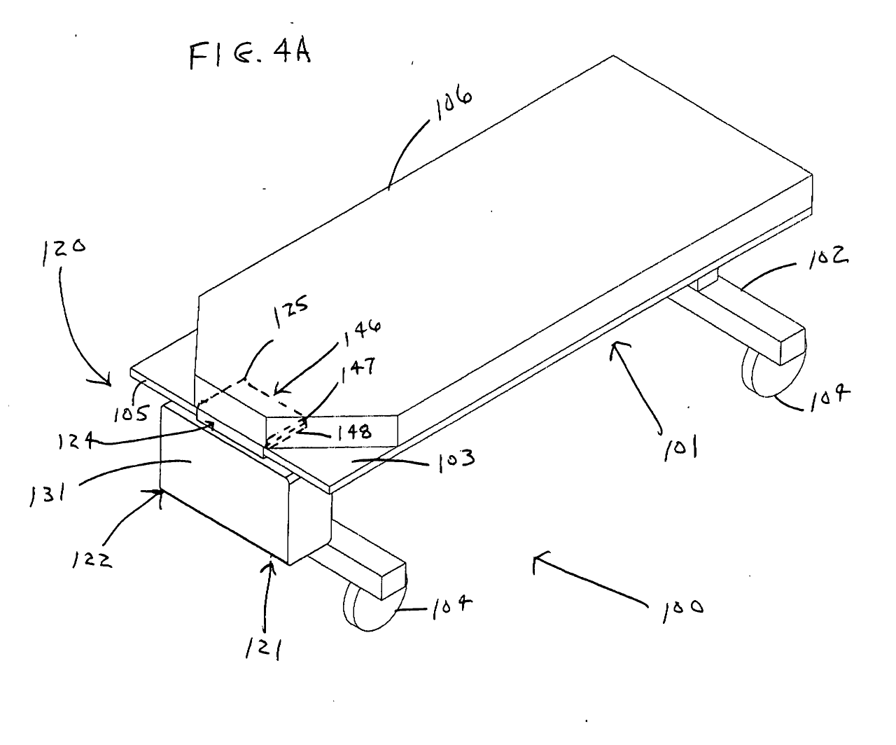

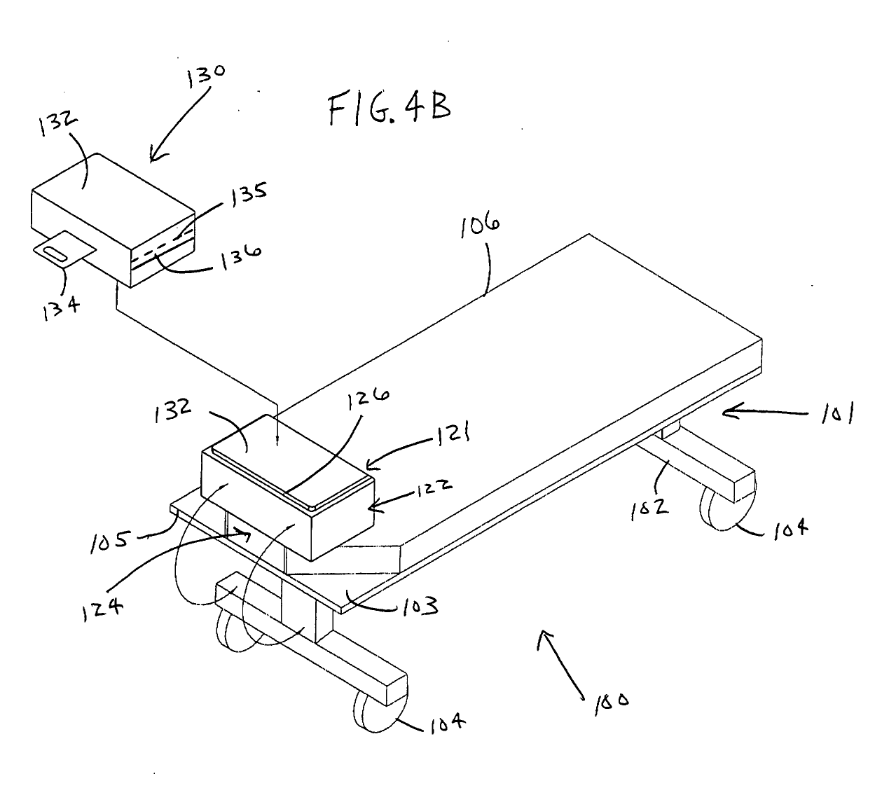

Further, the outer container 20 may be attached at various portions of the transport device 12. For example, the mountable outer container 20 may be attached at the head, under the back rest, at the foot or underside of a stretcher frame, or any other portion of any other transport device 12. Further, for example, in one embodiment the outer container 20 is mounted such that the outer container 20 is in a first state at least partially under a surface of a transport device 12 (e.g., see FIG. 4A) when mounted thereto and such that the closeable outer container 20 is repositionable to a second state at least partially above the surface of the transport device 12 (e.g., see FIG. 4B) such that the cavity 23 defined within the outer container 20 is accessible, for example, to a person positioning or retrieving a single use inner container 24 within or from the outer container 20.

The closure structure 26 for use in closing the opening 17 into cavity 23 may include any suitable mechanism for providing such closure. For example, the closure structure 26 may include a zipper with or without a lock, a mechanical latch with or without a lock, single use adhesive strips, hook and loop fasteners (e.g., Velcro), buttons, snaps, or any other type of closeable linkage.

Locking structure 28 used to lock the closure structure 26 to prevent access to the cavity 23 may include any suitable lock that can be associated with the closure structure 26 to prevent such access. For example, locking structure 28 may include a keylock, combination security seal (e.g., serialized or other), an electronic keypad lock, a combination lock, or any other lock where a person can be provided with a personalized mechanism for unlocking the outer container 20. For example, a keylock that locks only when a key is removed and which can be kept in possession of a patient until it is time to remove belongings or objects 15 from the interior cavity 23 of the outer container 20 may be used.

The single use inner container 24 may be formed of any suitable material such as, for example, those providing a sanitary condition for retaining objects belonging to a person. For example, the single use inner container 24 may be formed of an opaque plastic such that the objects can be seen, fiber materials, mesh materials, Tyvek-type material, etc.

The single use inner container 24 is provided with a tamper evident closure for sealing opening 19 after objects 15 belonging to a person are inserted therein. The tamper evident closure refers to any closure having a seal that when opened cannot be resealed without the appearance of a disruption to the seal. For example, a tamper evident closure may include an adhesive seal that includes a perforated tear strip or flap (e.g., one that may be pulled from any direction to break the seal and open the closure, much like a Federal Express package); a tamper evident adhesive; a flap element with perforations that can be form-fit to the outer container 20 for easy storing and removal; etc. In one or more other embodiments, closures that are not necessarily tamper evident may also be used, such as Ziploc closures, pressure sensitive adhesives, etc.

Further, the single use inner container 24 may include one or more other various features such as a carrying handle, a region thereon that can be written upon (e.g., the patient's ID may be written thereon), a pocket for receiving a card identifying the person to whom the objects within the single use inner container belong, and regions or portions attached or integral therewith that may include a serialized number that can be logged to a person whose belongings have been positioned within the single use inner container 24.

In addition, as shown in FIG. 1, the personal object security system 10 may include a secure attachment mechanism 30 that is secured at a first end 31 to the transport device 12 and at a second end 32 to the mountable container apparatus 11. The secure attachment mechanism 30 may include any mechanism that may be used to prevent a quick grab and run theft of the mounted outer container apparatus 20. For example, the secure attachment mechanism 30 may include a lanyard, a chain attaching mechanism, or any other mechanical securing device.

One embodiment of the mountable container apparatus 11 is designed to protect the staff, institution, and a patient from loss of personal belongings and patient privacy. For example, the mountable container apparatus 11 may be suitable for mounting to a stretcher or any patient transport device and is designed to provide additional security for patient belongings and information, therefore minimizing the potential for institutional liability for loss of a patient's personal items or compromised patient information privacy.

However, the present invention may also be used in one or more other applications, with or without being mounted to a transport device 12 (e.g., a container apparatus 11 that is not mounted and may or may not include a mounting structure 22). For example, such a container apparatus 11 that includes at least the outer container 20 and the single use inner container 24, with or without one or more of the associated features (e.g., locking structure, closure structure, etc.) may be used in applications such as in police station where objects of a person are typically secured, an airport security situation where a person's belongings may be secured for a period of time, or any other application where security of the belongings of a person are an issue.

One or more personal object security methods may be implemented using the personal object security system 10 generalized in FIG. 1. For example, one exemplary embodiment of a personal object security method 40 is shown in the block diagram of FIG. 2.

The personal object security method 40, as shown in FIG. 2, includes providing a mountable outer container 20 (block 42). The mountable outer container 20 is mounted to the transport device 12 (block 44). A single use inner container 24 is provided to a person (block 48). One or more objects belonging to the person are inserted into the single use inner container 24 (block 46). The single use inner container 24 is then inserted into the mountable outer container 20 (block 50). One skilled in the art will understand that the objects may be inserted into the single use inner container 24 before the single use inner container 24 is positioned within cavity 23 of the mountable outer container 20, or after insertion of the single use inner container 24 into the outer container 20.

After insertion of the single use inner container 24 into the mountable outer container 20 (block 50), a technique for securing access only to the person owning the belongings or objects inserted into the single use inner container 24 is employed (block 52). Such a secure access technique (block 52) generally includes locking the outer container 20 (block 54) and, thereafter, providing access to the interior cavity 23 of the locked outer container 20 to obtain the single use inner container 24 therefrom (block 56).

Once a person is given access to the single use inner container 24 (block 56), the single use inner container 24 can be visually inspected to determine if the inner container 24 has been compromised (block 58). For example, with use of the tamper evident closure on the single use inner container 24, if someone has tampered with the single use inner container 24, a visual inspection will provide notice that such tampering has occurred.

FIG. 3 shows one exemplary embodiment of a more detailed secure access technique 52 that may be used in implementing the personal object security method 40 as shown in FIG. 2. For example, the outer mountable container 20 may be locked using a keylock locking structure 28 (block 60). The key associated with the keylock is then secured with the patient whose belongings are within the single use inner container 24 positioned within the mountable outer container 20 (block 62). For example, any suitable wristband may be attached to the key which can be secured or placed on the patient's wrist or attached in some other fashion to the patient. When the patient whose belongings are within the single use inner container 24 desires access thereto, the patient provides the key for opening the keylock which is securing the outer mountable container 20 (block 64). Using the provided key, the patient can access the single use inner container 24 within the outer container 20 (block 66).

One will recognize that various other techniques for tracking the single use inner container 24 with the patient whose belongings are positioned therein may be used in combination with any other features described herein. For example, serialization of the single use inner containers and logging of such containers to patients may be used for tracking purposes. Further, various mechanisms for unlocking the outer container 20 may be secured with the patient in one or more various ways. For example, a card-type key may be attached to a patient by hook and loop fasteners, or otherwise secured to the person. Further, for example, the card may also include serialization data and/or patient identification data or other information.

In one particular exemplary embodiment, related to use of a mountable container apparatus 11 in a clinic or hospital setting, the outer container 20 may be a heavy duty vinyl pouch (e.g., similar to that of a backpack) that attaches securely and easily to an existing transport device (e.g., a stretcher). The heavy duty pouch may attach onto the stretcher frame, for example, under a backrest located at the head end of the stretcher, at the foot of the stretcher, or under the stretcher.

The pouch may open with a zipper and lock with a key locking device. A patient's personal articles and valuables can be stored in a single use tamper evident plastic liner that can be retained within the pouch. The liner may have an adhesive strip to seal an opening defined in the single use liner through which personal articles and valuables are inserted. Further, the liner may have an integrated carrying handle and a perforated easy opening tear strip (e.g., like a Federal Express package). Thus, this ensures a person's property remains within a tamper evident package while locked in the stretcher-attached pouch.

Further, in one such embodiment, a key for locking the pouch can only be removed when the lock is in a locked position. A wristband that holds the key may be placed on the patient's wrist throughout an examination or other procedure which required the use of the pouch. Upon completion of the examination, the patient may return the key to the attending nurse, who unlocks the pouch and returns the tamper evident plastic liner to the patient. Visual examination quickly determines if the liner has been compromised. In this particular embodiment, the key remains in the lock until the next process with the same or a different person.

Such an embodiment of the personal object security system 10 virtually eliminates the risk of a patient's personal property being lost, misplaced, soiled, or stolen. Further, the mountable container apparatus 11 eliminates the need for costly lockers that also consume valuable space. The mountable container apparatus 11 in the form of such a pouch is easily and securely attached to existing and conventional transport devices, such as stretchers, and further, with use of the single use, sanitary, tamper evident liner with easy carrying handle, a patient's personal property can be kept clean and secure before, during, and after an examination.

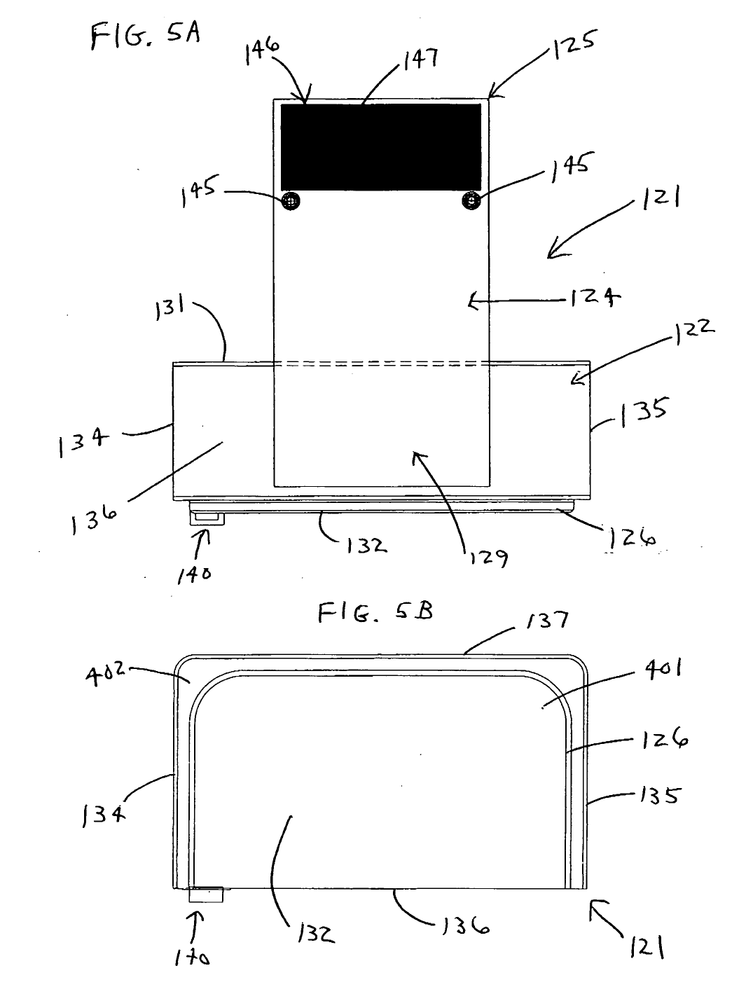

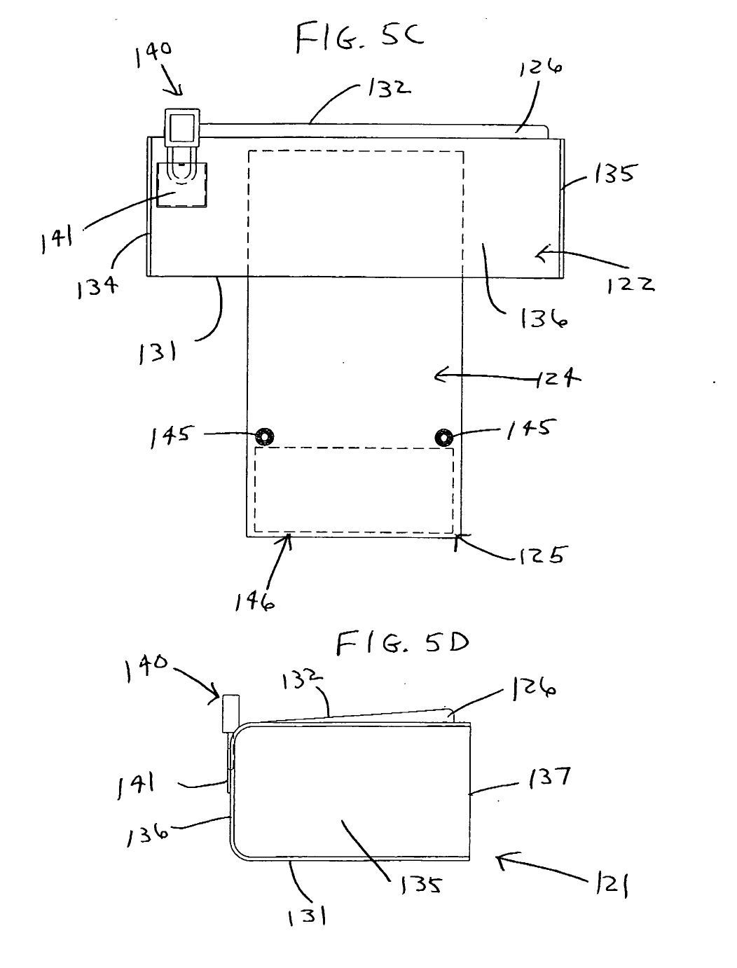

FIGS. 4A and 4B and FIGS. 5A-5D show one exemplary embodiment of a personal object security system 10 generalized in FIG. 1. The personal object security system 100, as shown in FIGS. 4A-4B, includes a stretcher 101 upon which a mountable container apparatus 120 is mounted. The stretcher 101 includes a stretcher frame 102 transportable by wheels 104. Stretcher frame 102 supports a bedding surface 103 upon which a pad 106 is positioned.

The mountable container apparatus 120 includes mountable outer container 121 which defines a cavity (not shown) in which a single use inner container 130 is received (see FIG. 4B). The single use inner container 130 includes a body of liner material 132 defining a cavity (not shown) and an opening 135 through which objects associated with a person may be inserted into the cavity. The body of liner material 132 is configured to be retained within the mountable outer container 121 when objects associated with the person are inserted into the cavity. The body of liner material 132 has associated therewith a tamper evident closure 136 for use in sealing the opening 135, as well as a handle 134.

The mountable outer container 121 is shown in further detail in FIGS. 5A-5D. Generally, the mountable outer container 121 includes a first body of material 122 defining a cavity (not shown) configured to receive the at least one single use inner container 130 and a second body of material 124 extending from the first body of material 122 to a distal end 125 thereof. FIG. 5A shows a first side view along the length of the outer container 121, FIG. 5B shows a top plan view of the outer container 121 with a second body of material 124 removed, FIG. 5C shows a second side view along a length of the outer container 121 opposite FIG. 5A, and FIG. 5D shows a third side view of the outer container 121 again with the second body of material 124 removed.

As shown in FIG. 5A, the outer container 121 includes the first body of material 122 which defines a cavity (not shown) in which the single use inner container 130 is received and the second body of material 124 extending therefrom. As shown in FIGS. 5A-5D, the first body of material 122 defining the cavity includes four sides 134-137, along with an access top panel 132 and a bottom panel 131. Generally, although outer container 121 may take various shapes, the outer container 121, as shown in FIGS. 5A-5D, is rectangular-shaped. The second body of material 124 (e.g., a rectangular-shaped panel) extends from a first end 129 attached to side panel 136 of the first body of material to a second distal end 125.

As shown in FIGS. 5A-5D, the outer container 121 includes a zipper 126 as the closure structure that allows the outer container 121 to be closed. In one particular embodiment, the zipper 126 is a three-sided circumfluent locking zipper (e.g., circumfluent with three sides of the four-sided (e.g., sides 134-137) outer container 121). The zipper 126 allows for securing closing material 401 associated with upper access panel 132 to the remainder portion 402 of the upper access panel 132.

As further shown in FIGS. 5A-5D, a keylock structure 140 is provided for locking the zipper 126 in a secure position after a single use inner container 130 is positioned within the cavity defined by the first body of material 122. The keylock structure 140 is provided with a pocket 141 for positioning purposes. One will recognize that various locking structures may be used.

As further shown in FIGS. 5A-5D, a mounting structure 146 is associated with the distal end 125 of the second body of material 124 for use in mounting the outer container 121 to the stretcher 101 or any other transport device. The configuration of the outer mountable container 120 shown in FIGS. 5A-5D allows the outer container 121 to be mounted to the stretcher 101 for ease of use and such that the mountable container apparatus 120 is not undesirably in the way of personnel using the stretcher 101.

As shown in FIGS. 4A and 4B, the outer container 121 is mounted using the mounting device 146 at the distal end 125 of the second body of material 124. The mounting device 146 is secured on the surface 103 of stretcher 101 such that the first body of material 122 (e.g., the rectangular body of material defining a cavity in which the single use inner container 130 is inserted) is positioned or allowed to hang off the edge 105 of the surface 103 and out of the way under the stretcher surface 103. In this first state (e.g., a stowed state), the bottom panel 131 of the first body of material 122 faces outward from the stretcher 101.

In one exemplary embodiment, the mounting device 146 includes a first hook and loop fastener portion 147 attached at the distal end 125 of the second body of material 124. The size of such a hook and loop fastener portion 147 is dependent upon the necessary load to be borne thereby. A second hook and loop fastener portion 148 is mounted on the surface 103, as shown in FIG. 4A. In such a manner, the first hook and loop fastener portion 147 is attached to the second hook and loop fastener portion 148 mounted on surface 103, and the mountable outer container 121 is attached such that it can be stowed below surface 103. One will recognize that the length of the second body of material 124 (e.g., the strap) attaching the first body of material 122 to the stretcher 101 is long enough to allow the bag to hang out of the way, under the stretcher surface 103. However, the length of the second body of material 124 must be also short enough such that the outer container 121 does not drag on the surface over which the stretcher 101 is being transported.

With the outer container mounted on stretcher surface 103, as described with reference to FIG. 4A, the outer container 121 can be flipped into a second state (e.g., an access state) for unloading or loading of objects into the cavity defined by the outer container 121. In this access position, the top access panel 132 is presented to a user such that zipper 126 is accessible. The strap or second body of material 124 is long enough to permit the outer container 121 to be flipped onto the pad 106 or another, for example, flat or otherwise configured surface (e.g., for loading and unloading). In other words, the outer container 121 is flipped into a position for access to zipper 126 and to allow the outer container 121 to be opened (e.g., unlocking and/or unzipping). The single use inner container 130 can then be removed from or loaded into the cavity defined by the outer container 121.

As further shown in FIGS. 5A and 5C, grommets 145 are formed or otherwise associated with the second body of material 124. Such grommets 145 provide a location for implementing a secure attachment mechanism 30, as shown generally in FIG. 1. For example, the grommets 145 allow for use of a lanyard to be inserted through one or more of the openings formed by the grommets 145 and then be attached to frame 102 of stretcher 101 for additional security. However, any other additional attachment mechanism may be used to provide security for preventing a grab and run situation.

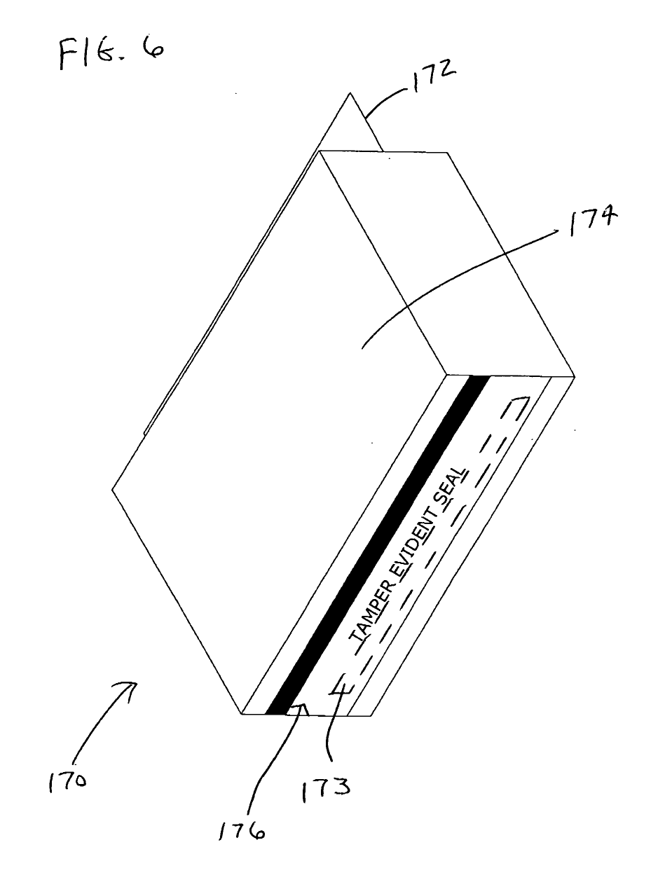

FIG. 6 shows an alternate embodiment of a single use inner container as shown generally in FIG. 1. The single use inner container 170 of FIG. 6 includes a body of liner material 174 which, when in its expanded state, takes a generally rectangular form. The single use inner container 170 shown in FIG. 6 is in a configuration such that it has objects positioned therein.

The body of liner material 174 generally defines a cavity in which the objects may be placed through an opening 173. The opening 173 may be closed by way of a tamper evident closure 176 which extends adjacent the opening 173. The tamper evident closure 176, as shown in FIG. 6, includes an adhesive seal with a perforated tear strip for opening the single use inner container 170. Further, the body of liner material 174 includes handle structure 172 extending therefrom for ease of use.

FIG. 7 shows a top view of an unused single use inner container such as that for use in implementing the personal object security system 10 shown generally in FIG. 1. The single use inner container 180, as shown in FIG. 7, includes a rectangular two-panel body of material 183 (only the top panel being shown in FIG. 7). The panels extend from a first end 181 to a second end 182.

An opening 187 is defined at the first end 181 for insertion of objects within the body of material 183. The opening 187 is shown as a dotted line opening as it is on the reverse side or bottom panel (not shown) of the two panel body of material 183.

A covered seal tape 190 is formed at the first end 181 of the body of material 183 and adjacent to the opening 187. A mating surface 192 for the seal tape is positioned such that when the tape backing of the covered seal tape 190 is removed and the seal tape 190 is positioned on the mating surface 192, then the opening 187 is sealed and cannot be opened and resealed without compromising the seal in one manner or another. A perforated tear strip 188 is provided along the width of the liner material 183 at the first end 181 for ease in opening the sealed single use inner container 180.

As further shown in FIG. 7, an opening 184 is provided through both the top and bottom panels of the body of liner material 183 so as to form a handle for carrying purposes. The opening 184 is formed at the second end 182 opposite the opening 187.

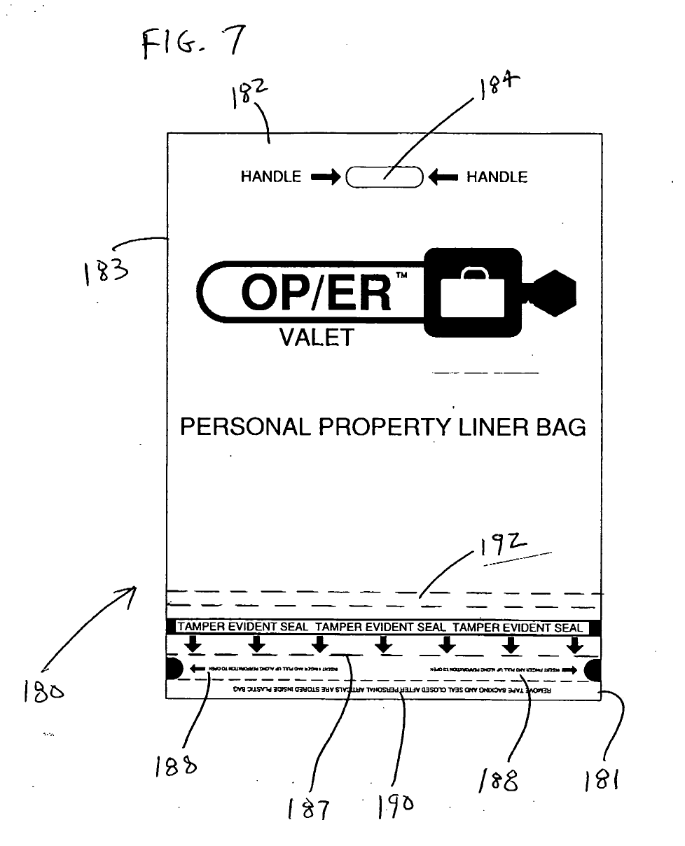

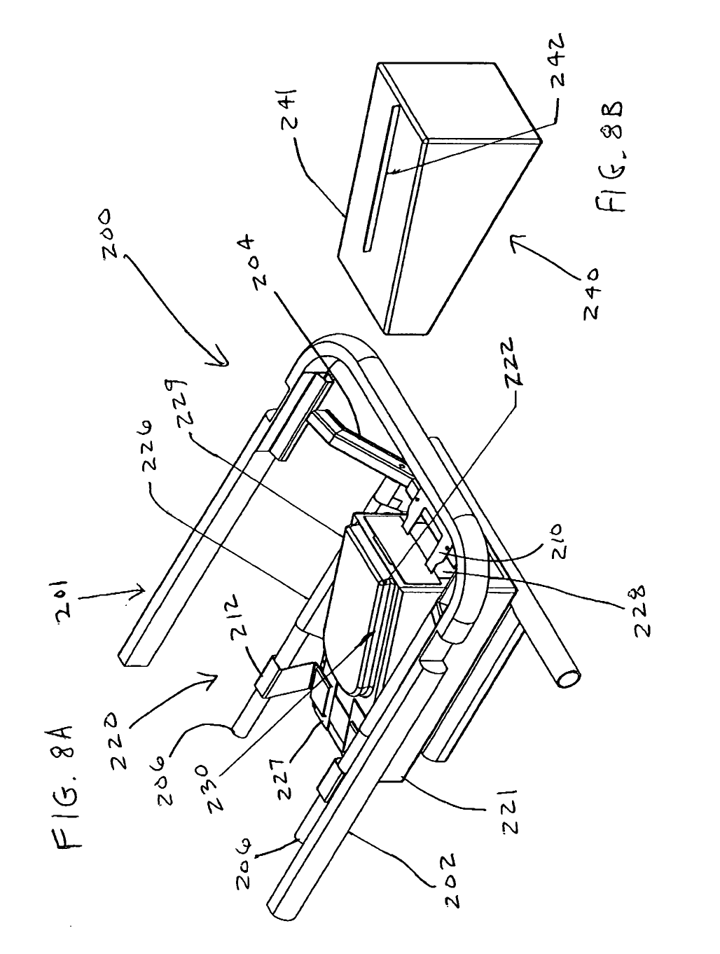

FIGS. 8A and 8B show an alternative embodiment of a personal object security system such as that shown generally in FIG. 1. The personal object security system 200 includes a mountable container apparatus 220 mounted on a gurney 201. The mountable container apparatus 220 includes an outer container 221 (as shown in FIG. 8A) that defines a cavity (not shown) for receiving removable sealed plastic inner liner 240 as shown in FIG. 8B. The removable inner liner 240 defines a cavity 241 for receiving objects through an opening 242 thereof. A tamper evident closure is provided for use with the removable inner liner 240.

The gurney 201 includes a frame structure 202. The frame structure 202 includes frame tubes 206 generally running longitudinally with the gurney length. Further, other frame structure 204 is provided across frame structure 202 for providing support for the structure.

The outer container 221 is formed by a body of material 222 having a plurality of sides, one of which is a top panel 229 that includes a zipper 230 for providing access into the interior of the outer container 221. Various attachment flaps 226, 227, 228 are provided for mounting the outer container 221 to the gurney 201. For example, attachment flaps 226 are fastened using Velcro to frame tubes 206, attachment strips 227 are used for mounting outer container 221 to a bracket 212 positioned between the frame tubes 206, and attachment flap 228 is used for mounting the outer container 221 using bracket 210 bolted to the gurney frame 202. One skilled in the art will recognize that various attachment mechanisms may be used to mount the outer container 221 and that the types of attachment flaps and locations thereof may vary.

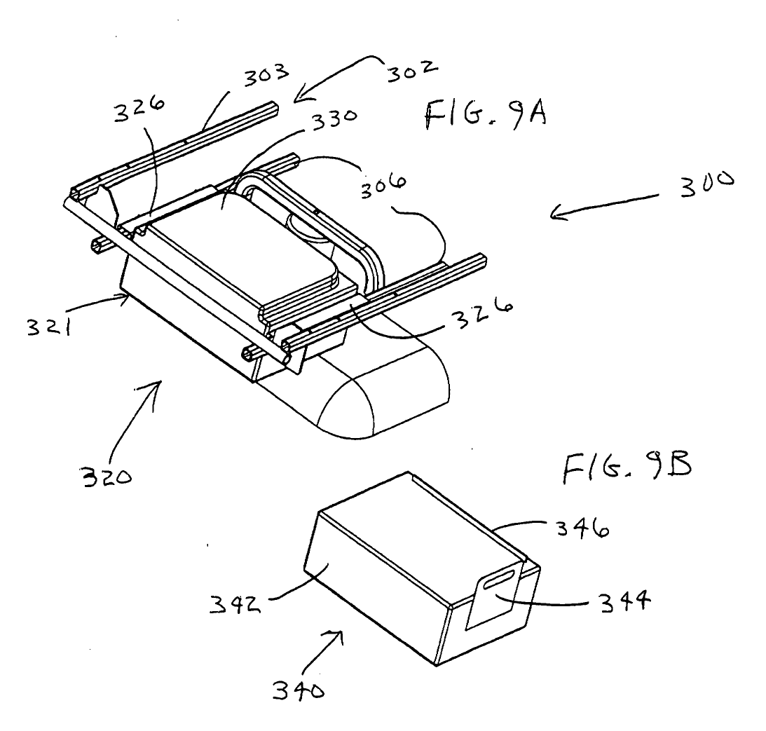

FIGS. 9A and 9B show yet another alternative embodiment of a personal object security system such as that shown generally in FIG. 1. The personal object security system 300 includes a mountable container apparatus 320 (as shown in FIG. 9A) including an outer container 321 mounted to a gurney 302. The outer container 321 defines a cavity in which a single use inner container 340 (as shown in FIG. 9B) is received.

The single use inner container 340 includes a clean, disposable and security sealable, plastic liner bag 342 which can be inserted and received into outer container 321. The single use inner container 340 includes an opening 346 through which objects may be inserted and which is closed by a tamper evident closure. Further, the inner container 340 includes a handle 344 attached or otherwise associated therewith.

The gurney 302 includes a frame structure 303 comprising framing tubes 326 which run in the longitudinal direction along the length of the gurney 302. Various framing structures are used to support the longitudinally running framing tubes 326, as shown in FIG. 9.

The outer container 321 includes a plurality of sides for defining a cavity in which the single use inner container 340 may be received. One of such sides of the outer container 321 includes a locking peripheral zipper 330 which can be opened for access to the interior of the outer container 321. The outer container 321 is mounted to the longitudinally extending framing tubes 306 by attachment flaps 326. The attachment flaps 326 are wrapped around the framing tubes 306 and then attached back to the outer container 321 using hook and loop fasteners (e.g., Velcro).

One will recognize that various locations of the transport devices may be used for positioning of the outer containers as shown in FIGS. 8 and 9. For example, as shown in FIG. 9A, the mountable outer container 321 is mounted beneath a structure that would support a person lying on the gurney (the structure surfaces and pads used to support such a person being removed to show the location of the outer container 321). The same applies to the structure for supporting a person in the embodiment of FIG. 8.

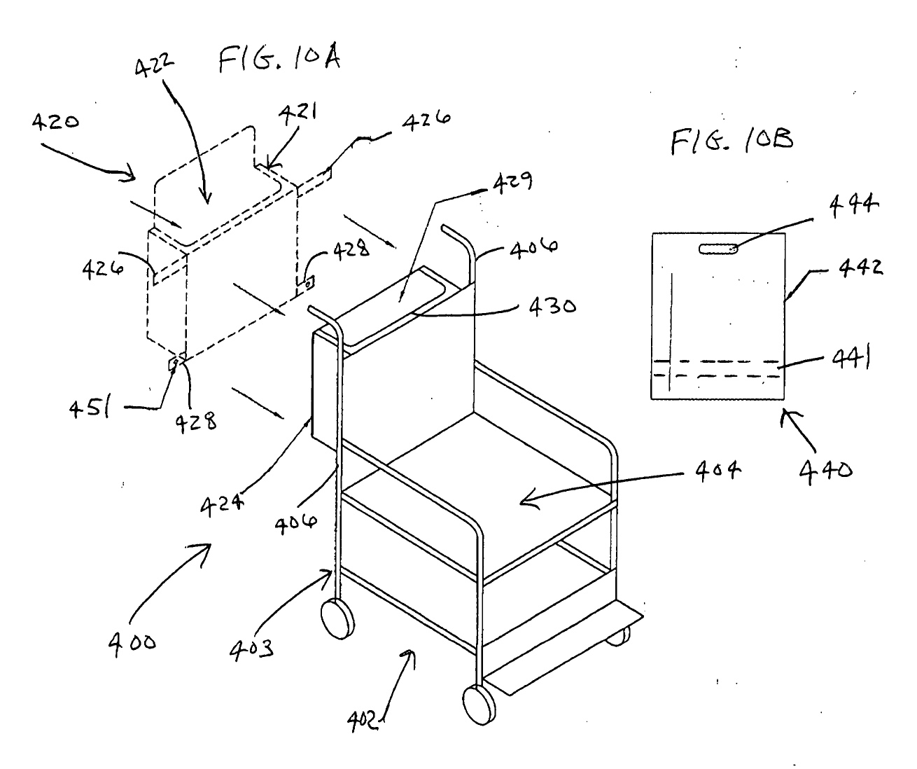

FIGS. 10A and 10B show an alternative embodiment of a personal object security system such as that shown generally in FIG. 1 used with a wheelchair 402. The personal object security system 400 includes a mountable container apparatus 420 mounted on the wheelchair 402, and also separated from the wheelchair 402 as shown by the dashed line drawing of the mountable container apparatus 420 in FIG. 10A.

The mountable container apparatus 420 includes an outer container 421 (as shown in FIG. 10A) that defines a cavity 422 for receiving a removable liner bag 440 as shown in FIG. 10B. The removable inner liner bag 440 includes a body of material 442 that defines a cavity therein (not shown) for receiving objects through an opening defined therein. A tamper evident closure 441 is provided for use with the removable inner liner 440 and a handle 444 is also defined in the body of material 442.

The wheelchair 402 includes a frame structure 403 that defines a seat 404 in which a person may be seated. The frame structure 403 includes a plurality of frame tubes 406 (e.g., generally running parallel to one another) along the back side of the wheelchair 402. Further, other frame structure of the wheelchair 402 provides other functionality (e.g., frame that supports the person seated, frame structure that supports the wheels of the chair, etc.). Although the mountable container apparatus 420 is described as being mounted to the back of the wheelchair 402 employing framing associated with the frame tubes 406, one skill in the art will recognize that the mountable container apparatus 420 may be mounted on other parts of the wheelchair 402 as well (e.g., below the seat 404 of the wheelchair 402, along the side of the wheelchair 402, etc.).

The outer container 421 is formed of a body of material 424 having a plurality of sides (e.g., formed in a rectangular shape, shaped liked backpack, etc.). One of the sides is a top panel 429 that includes a zipper closure 430 for providing access into the cavity 422 (e.g., interior) of the outer container 421. A locking feature such as described with reference to FIG. 1 may also be employed with the zipper closure 430.

Various attachment structure 426 and 428 are provided for mounting the outer container 421 to the wheelchair 402 (e.g., hook and loop fasteners wrapping about the framing structure, additional brackets for supporting the outer container 421, etc.). One skilled in the art will recognize that various attachment mechanisms may be used to mount the outer container 421 and that the types of attachment structure 426, 428 will vary, as will the locations thereof.

Yet further, an additional secure attachment mechanism may be used to prevent a quick grab and run theft of the mountable outer container 421. For example, the secure attachment mechanism may include grommets 451 defining openings through which a lanyard may be inserted and secured to the wheelchair 402.

All patents, patent documents, and references cited herein are incorporated in their entirety as if each were incorporated separately. This invention has been described with reference to illustrative embodiments and is not meant to be construed in a limiting sense. As described previously, one skilled in the art will recognize that other various illustrative applications may use the techniques as described herein to take advantage of the beneficial characteristics of the apparatus, methods, and systems described herein (e.g., sports facilities, nursing home facilities, police stations, prisons, etc.). Various modifications of the illustrative embodiments, as well as additional embodiments to the invention, will be apparent to persons skilled in the art upon reference to this description.

1. A container apparatus for use with a transport device, the container apparatus comprising:
a closable outer container mountable to the transport device; and
at least one single use inner container configured to be retained within the outer container, wherein the at least one single use inner container defines an opening through which objects associated with a person may be inserted into the single use inner container, and further wherein the at least one single use inner container comprises a tamper evident closure for use in sealing the opening.

2. The container apparatus of claim 1, wherein the closable outer container is lockable to secure the at least one single use inner container within the closable outer container.

3. The container apparatus of claim 2, wherein the container apparatus comprises a key lock for locking the closable outer container, wherein the key lock will lock only when a key thereof is removed.

4. The container apparatus of claim 1, wherein the closable outer container comprises:
a first body of material defining a cavity configured to receive the at least one single use inner container;
a second body of material extending from the first body of material to a distal end thereof; and
a mounting device associated with the distal end of the second body of material for use in mounting the closable outer container to the transport device.

5. The container apparatus of claim 4, wherein the mounting device comprises a first portion of a hook and loop fastener at the distal end of the second body of material configured for attachment to a second portion of a hook and loop fastener mountable on the transport device.

6. The container apparatus of claim 4, wherein the mounting device associated with the distal end of the second body of material is configured to position the closable outer container in a first state at least partially under a surface of a transport device when mounted thereto, and further is configured to allow the closable outer container to be repositioned to a second state at least partially above the surface of the transport device such that the cavity defined by the first body of material is accessible.

7. The container apparatus of claim 1, wherein the apparatus further comprises an attachment mechanism adapted to secure the closable outer container to the transport device after the closable outer container is mounted thereto.

8. The container apparatus of claim 1, wherein the at least one single use inner container comprises a handle.

9. A method for securing objects belonging to a person for use with a transport device, the method comprising:
providing a closable outer container mountable to the transport device;
inserting the objects of the person into at least one single use inner container configured to be retained within the closeable outer container, wherein the at least one single use inner container comprises a tamper evident closure for use in sealing the single use inner container; and
inserting the at least one single use inner container within the closeable outer container.

10. The method of claim 9, wherein the method further comprises locking the closable outer container.

11. The method of claim 10, wherein locking the closable outer container comprises providing a key lock for locking the closable outer container, wherein the key lock will lock only when a key thereof is removed.

12. The method of claim 9, wherein the method further comprises mounting the closable outer container onto the transport device.

13. The method of claim 12, wherein the closable outer container comprises a first body of material defining a cavity configured to receive the at least one single use inner container and a second body of material extending from the first body of material to a distal end thereof, and further wherein mounting the closable outer container onto the transport device comprises:
positioning a first portion of a hook and loop fastener on a surface of the transport device; and
attaching a second portion of a hook and loop fastener associated with the distal end of the second body of material to the first portion of the hook and loop fastener.

14. The method of claim 12, wherein mounting the closable outer container onto the transport device comprises mounting the closable outer container onto the transport device such that the closable outer container is locatable in a first state at least partially under a surface of a transport device when mounted thereto and such that the closable outer container is repositionable to a second state at least partially above the surface of the transport device such that a cavity defined within the closable outer container is accessible.

15. The method of claim 14, wherein mounting the closable outer container onto the transport device comprises mounting the closable outer container onto the transport device such that the closable outer container is repositionable to the second state resting on the surface of the transport device and the cavity is accessible.

16. The method of claim 12, wherein mounting the closable outer container onto the transport device further comprises securing the closable outer container to the transport device after the closable outer container is mounted thereto.

17. A liner apparatus for use with a container apparatus mountable onto a transport device, the liner apparatus comprising:
a body of liner material defining a cavity and an opening through which objects associated with a person may be inserted into the cavity, wherein the body of liner material is configured to be retained within the mountable container apparatus when objects associated with a person are inserted into the cavity; and
a tamper evident closure for use in sealing the opening.

18. The liner apparatus of claim 17, wherein the liner apparatus further comprises a handle.

19. The liner apparatus of claim 17, wherein the tamper evident closure comprises an adhesive seal tape and a perforated tear strip.
